(19) United States
(12) Patent Application Publication
(10) Pub. No.: US 2025/0367014 A1
(43) Pub. Date: Dec. 4, 2025

(54) COMPACT, FULL-RANGE OF MOTION AND MULTI-DEGREE OF FREEDOM STRUCTURE FOR SUPPORTING ORTHOTIC DEVICES

(71) Applicant: B-TEMIA INC., St-Augustin-de-Desmaures (CA)

(72) Inventors: Marcus BROOKSHAW, St-Augustin-de-Desmaures (CA); Stéphane BÉDARD, St-Augustin-de-Desmaures (CA); Jean-Philippe CLARK, St-Augustin-de-Desmaures (CA); Nathaniel ZOSO, St-Augustin-de-Desmaures (CA)

(21) Appl. No.: 18/870,931

(22) PCT Filed: Jun. 1, 2023
(86) PCT No.: PCT/CA2023/050754

§ 371 Date: Dec. 2, 2024

Related U.S. Application Data
(60) Provisional application No. 63/348,211, filed on Jun. 2, 2022.

(51) Int. Cl. A61F 5/01

(52) U.S. Cl. CPC A61F 5/0123; A61F 5/013; A61F 2005/0134; A61F 2005/0137

(57) ABSTRACT

A compact, multi-Degree of Freedom (DoF) support structure located within a single biomechanical plane, for supporting an orthotic device allowing for a user's full range of motion in all biomechanical plans, The support structure comprises a support belt configured to be secured around the user's body and a multi-DoF motion element that includes a frontal plane rotational DoF mechanism, a transverse plane rotational DoF mechanism and a sagittal plane rotational DoF mechanism, allowing the multi-Degree of Freedom (DoF) support structure to support the orthotic device while allowing for the user's full range of motion in all biomechanical plans.

### CROSS-REFERENCE TO RELATED APPLICATIONS

This application claims the benefits of U.S. provisional patent application No. 63/348,211 filed on Jun. 2, 2022, which is herein incorporated by reference.

### TECHNICAL FIELD

The present disclosure relates to a compact, full Range of Motion (RoM) and multi-Degree of Freedom (DoF) structure for supporting orthotic devices.

### BACKGROUND

Hip-aligned devices like exoskeletons and orthoses that connect above and below the waist are used to provide structural and mechanical assistance for a variety of user needs but come at the cost of reduced user hip mobility. This is a consequence of the natural anatomy of human hips and need for the attached devices to remain aligned with the axes of the human hip without losing the ability to transmit forces. Solutions involving large mechanical arrangements or the sacrifice of one or more natural axes of hip mobility (typically abduction/adduction and/or internal/external rotation) exist but come at the cost of wearing a heavy/cumbersome or mobility-limiting device.

Furthermore, traditional lower-body exoskeletons with hip and knee actuators tend to assist the hip motion in the sagittal plane with hip actuators. Hip movements in the frontal and horizontal plane are allowed by degrees of freedom that align with the user's hip joint axis. Off-axis degrees of freedom are generally avoided because the axis displacement induces a mechanical constraint on the thigh segment when the user moves the hip outside of the sagittal plane.

Accordingly, there is a need for a lightweight, low-profile solution that can align an exoskeleton/orthosis and allow comfortable force transmission across the hip joint without limiting the user's natural joint mobility.

### SUMMARY

The present disclosure provides a compact, multi-Degree of Freedom (DoF) support structure located within a single biomechanical plane, for supporting an orthotic device allowing for a user's full range of motion in all biomechanical plans, the support structure comprising:
a support belt configured to be secured around the user's body, connected to a multi-DoF motion element through a first interconnection, the multi-DoF motion element including a first, a second and a third rotational DoF mechanisms, the first rotational DoF mechanism being connected to the second rotational DoF mechanism through a second interconnection, and the second rotational DoF mechanism being connected to the third rotational DoF mechanism through a third interconnection, each of the first, second and third rotational DoF mechanisms being uniquely selected from a group consisting of a frontal plane rotational DoF mechanism, a transverse plane rotational DoF mechanism and a sagittal plane rotational DoF mechanism;
the multi-DoF motion element further including an attachment mechanism for connecting thereto an orthotic device;
wherein in use the compact, multi-Degree of Freedom (DoF) support structure supports the orthotic device while allowing for the user's full range of motion in all biomechanical plans.

The present disclosure also provides a compact, multi-Degree of Freedom (DoF) support structure wherein the support belt includes a zero-rigidity material, an infinite rigidity material or a material having a rigidity different than zero and infinity.

The present disclosure further provides a compact, multi-Degree of Freedom (DoF) support structure wherein the support belt includes a material selected from the group consisting of fabric, foam, plastic and metal.

The present disclosure also further provides a compact, multi-Degree of Freedom (DoF) support structure wherein the frontal rotational DoF mechanism, the transverse rotational DoF mechanism and the sagittal rotational DoF mechanism take the form of a mechanism selected from the group consisting of a pivot, a hinge, a passive rotational mechanism and an active rotational mechanism.

The present disclosure still further provides a compact, multi-Degree of Freedom (DoF) support structure wherein the frontal rotational DoF mechanism, the transverse rotational DoF mechanism and the sagittal rotational DoF mechanism include a zero-rigidity material, an infinite rigidity material or a material having a rigidity different than zero and infinity.

The present disclosure also provides a compact, multi-Degree of Freedom (DoF) support structure wherein the frontal rotational DoF mechanism, the transverse rotational DoF mechanism and the sagittal rotational DoF mechanism include a material selected from the group consisting of fabric, foam, plastic and metal.

The present disclosure further provides a compact, multi-Degree of Freedom (DoF) support structure wherein the orthotic device is a passive orthosis or a powered orthosis.

The present disclosure also further provides a compact, multi-Degree of Freedom (DoF) support structure wherein the third rotational DoF mechanism is a sagittal plane rotational DoF mechanism, and the attachment mechanism is an off-axis sagittal plane translational DoF mechanism passively linking the third rotational DoF mechanism to the orthotic device, wherein the off-axis sagittal plane translational DoF mechanism is a self-adjusting variable length structure allowing a variation of distance between the third rotational DoF mechanism and the orthotic device so as to prevent misalignment from imposing physical constraints on the movement of the biological joints.

The present disclosure still further provides a compact, multi-Degree of Freedom (DoF) support structure wherein the off-axis sagittal plane translational DoF mechanism is in the form of a prismatic joint or a cylindrical joint.

The present disclosure also provides a compact, multi-Degree of Freedom (DoF) support structure wherein the orthotic device includes a joint selected from the group consisting of an ankle joint, a knee joint, a hip joint, a wrist joint, an elbow joint and a shoulder joint, aligned with a corresponding user's joint.

The present disclosure further provides a compact, multi-Degree of Freedom (DoF) support structure wherein the support belt is configured to be secured around the user's torso or waist above the hip joint, around the user's shoulders above the shoulder joint, around the user's leg above the knee joint or around the user's arm above the elbow joint.

### BRIEF DESCRIPTION OF THE FIGURES

Embodiments of the disclosure will be described by way of examples only with reference to the accompanying drawings, in which:

FIG. 1 is a schematic side view of the compact, full Range of Motion (ROM) and multi-Degree of Freedom (DoF) structure in accordance with an illustrative embodiment of the present disclosure;

FIG. 2 is a schematic side view of a first alternative embodiment of the full Range of Motion (ROM) and multi-Degree of Freedom (DoF) hip structure;

FIG. 3 is a perspective view of the full Range of Motion (ROM) and multi-Degree of Freedom (DoF) hip structure of FIG. 2;

FIG. 4 is a front perspective view of the 2DoF motion element of the full Range of Motion (ROM) and multi-Degree of Freedom (DoF) hip structure of FIG. 2;

FIG. 5 is a schematic side view of a second alternative embodiment of the full Range of Motion (ROM) and multi-Degree of Freedom (DoF) hip structure;

FIG. 6 is a perspective view of the full Range of Motion (ROM) and multi-Degree of Freedom (DoF) hip structure of FIG. 5;

FIG. 7 is a schematic side view of a third alternative embodiment of the full Range of Motion (ROM) and multi-Degree of Freedom (DoF) hip structure, and

FIGS. 8A and 8B are perspective views of an alternative embodiment of the attachment mechanism in the form of an off-axis sagittal plane translational DoF mechanism, shown in a first (FIG. 8A) and a second (FIG. 8B) limb positions.

Similar references used in different Figures denote similar components.

### DETAILED DESCRIPTION

Generally stated, the non-limitative illustrative embodiment of the present disclosure provides a compact, full Range of Motion and multi-Degree of Freedom (DoF) support structure located within a single biomechanical plane, for supporting an orthotic device, for example a brace, an orthosis or an exoskeleton having a lower-body component, allowing for a user's full range of hip motion depending on a direction of motion. Advantageously, the support structure is sufficiently rigid so as to transmit hip joint movement to the user's hip in the sagittal plane (i.e., hip flexion/extension movements) while allowing for maximal freedom of motion in the other user's hip movements (i.e., hip internal/external rotation, and hip abduction/adduction), including the translational movements between the pelvis and knee joints.

The hip support structure further provides an off-axis linking structure resulting in a hip structure that is fully rigid in the sagittal plane in order to allow the hip motion to be entirely transmitted to the orthotic structure hip joint or actuator, while offering 2DoF to the user to allow movement in the horizontal (internal/external rotation) and frontal (abduction, adduction) planes. The axis of rotation of those 2DoF does not require to be aligned with the user's hip axis of rotation. In the case where the hip support structure is attached to a lower-body orthosis or exoskeleton without a knee joint, the resulting effect of this axis displacement is simply a sliding up or down of the thigh structure along the thigh. In the event the orthosis or exoskeleton is outfitted with a knee joint or actuator that requires being aligned with the user's knee joint, the hip structure is outfitted with a sliding femoral shaft, that allows removing the mechanical constraint generated by this axis displacement.

It is to be understood that the hip support structure is configured for use with orthotic devices or exoskeletons comprising a lower-body component, and which may or may not further comprise an upper-body component.

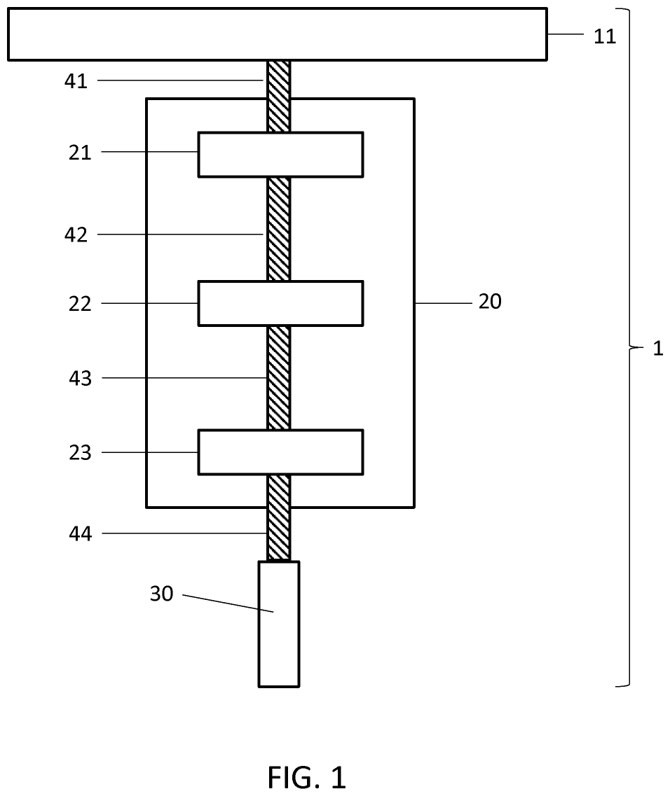

Referring to FIG. 1, the compact, full Range of Motion (ROM) and multi-Degree of Freedom (DoF) hip structure (1) includes a support belt (11) configured to be positioned around a user's torso or waist, to which is laterally secured in the sagittal plane by at least one interconnection (41). Interconnection (41) connects to a multi-DoF motion element (20) including a frontal plane rotational DoF mechanism (21), a transverse plane rotational DoF mechanism (22), and a sagittal plane rotational DoF mechanism (23). These DoF mechanisms (21, 22, 23) are interconnected by interconnections (42) and (43). The order of the DoF mechanisms (21, 22, 23) is not important, for example, any of the three DoF mechanisms (21, 22, 23) is connected to the support belt (11) through interconnection (41), any of the two remaining DoF mechanisms is connected to the first DoF mechanism through interconnection (42), and the remaining DoF mechanism is connected to the second DoF mechanism through interconnection (43). The multi-DoF motion element (20) is itself configured to be connected to an orthotic device (30) through an attachment mechanism (44), which can be, for example, a hip joint.

The frontal plane rotational DoF mechanism (21) allows motion of the orthotic device (30) around the abduction/adduction axis. The transverse plane rotational DoF mechanism (22) allows motion of the orthotic device (30) around the internal/external rotation axis. As for the sagittal plane rotational DoF mechanism (23), it allows motion of the orthotic device (30) around the flexion/extension axis. In an illustrative embodiment, the sagittal plane rotational DoF mechanism (23) can be the hip joint of the associated orthotic device (30).

It is to be understood that the DoF mechanisms (21, 22, 23) can be independently made from free hinges and pivots, or from flexible materials such as plastics, foams, polymers, and fabrics. The thinness of the flexible materials combined with their resilience allow motion of the orthotic device (30) around the abduction/adduction axis through bending and around the internal/external rotation axis through twisting, while restricting motion in the flexion/extension axis.

In a further illustrative embodiment, the attachment mechanism (44) is a sagittal plane translational DoF mechanism which compensates for misalignments between the user's joints and the orthotic device (30) joints generated by the fact that the DoF mechanisms (21, 22, 23) are not co-axial with the user's natural joints, thus preventing the orthotic device's (30) misalignment in this region from imposing physical constraints on the movement of the biological joints of the user. Accordingly, the user's leg is not impeded in its motion.

In another illustrative embodiment, the DoF mechanism (23) is the orthotic device (30) hip joint, and the sagittal plane translational DoF mechanism (44) links the orthotic device hip joint (23) to a knee and/or hip orthotic device.

It is to be understood that this compact, full Range of Motion (ROM) and multi-Degree of Freedom (DoF) structure (1) can be used at a user's hip or at other joints, for example the shoulders, elbows, wrists, knees and ankles, wherein the sagittal plane translational DoF mechanism (44) links a first joint (23) of an orthotic device (30) aligned with the user's proximal joint to a second joint or lateral segment of the orthotic device (30) aligned with the user's distal joint, thus preventing the orthotic device's (30) misalignment in this region from imposing physical constraints on the movement of the biological joints. Accordingly, the user's limb (leg or arm) is not impeded in its motion.

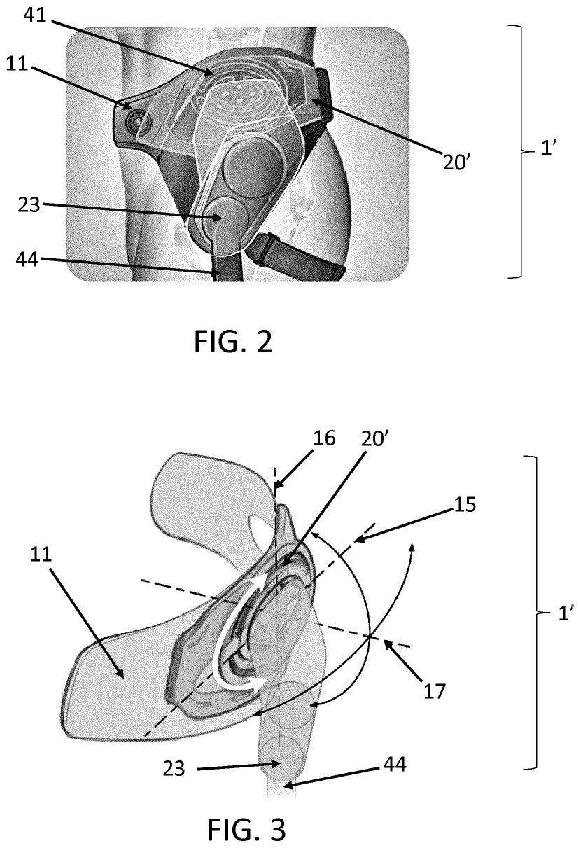

Referring to FIGS. 2 and 3, there is shown a first alternative embodiment of the full Range of Motion (ROM) and multi-Degree of Freedom (DoF) hip structure (1′), which includes a 2DoF motion element (20′) aligned with the user's hip joint center of rotation in the sagittal plane and secured on support belt (11) configured to be positioned around a user's torso or waist.

The 2DoF motion element (20′) has an attachment mechanism (44) positioned thereon to attach thereto the orthotic device (30). The 2DoF motion element (20′) allows motion of the attachment mechanism (44) around the abduction/adduction axis (15) as well as around the internal/external rotation axis (16) of the user's hip while restricting motion in the flexion/extension axis (17). Thus, the allowance or restraint of motion of the user's hip motion depends on the direction of motion.

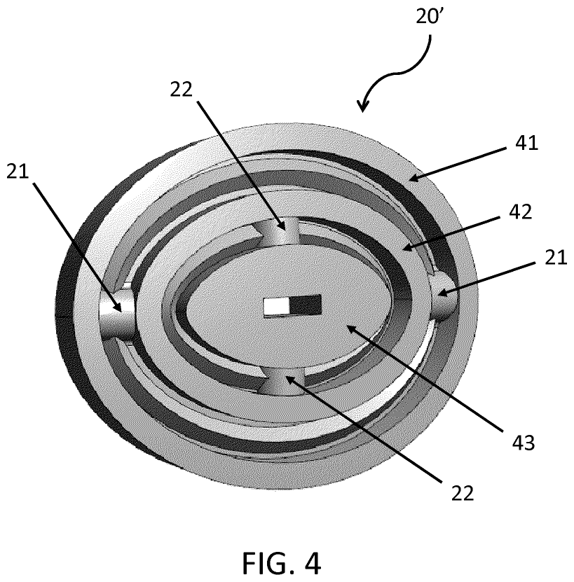

Referring to FIG. 4, the 2DoF motion element (20′) takes the form of a gimbal with interconnection (41) in the form of a fixed outer ring and pivotable interconnections (42) and (43) in the form of a middle ring and an inner ring, respectively. Interconnection (41) is secured to the support belt (11) to restrict motion in the flexion/extension axis (17) (see FIG. 3). The frontal plane rotational DoF mechanism (21), in the form of a first set of pivots, is pivotably connected between interconnection (41) and interconnection (42), allowing motion of the attachment mechanism (44) around the abduction/adduction axis (15) of the user's hip (see FIG. 3). The transverse plane rotational DoF mechanism (22), in the form of a second set of pivots, is pivotably connected between interconnection (42) and interconnection (43), allowing motion of the attachment mechanism (44) around the internal/external rotation axis (16) of the user's hip (see FIG. 3).

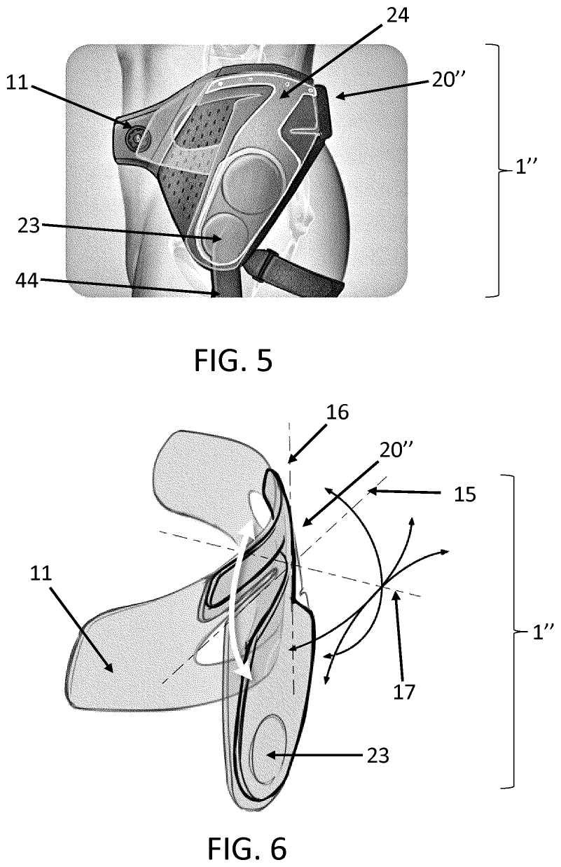

Referring to FIGS. 5 and 6, there is shown a second alternative embodiment of the full Range of Motion (ROM) and multi-Degree of Freedom (DoF) hip structure (1″), which includes a 2DoF motion element (20″) aligned with the user's hip joint center of rotation in the sagittal plane and secured on support belt (11) configured to be positioned around a user's torso or waist.

The 2DoF motion element (20″) has an attachment mechanism (44) positioned thereon to attach a lower body orthotic device (30) thereto. The 2DoF motion element (20″) allows motion of the attachment mechanism (44) around the abduction/adduction axis (15) as well as around the internal/external rotation axis (16) of the user's hip while restricting motion in the flexion/extension axis (17). Thus, the allowance or restraint of motion of the user's hip motion depends on the direction of motion.

The thinness of the upper portion 24 of the 2DoF motion element 20″ combined with the resilience of the materials used in the construction of the 2DoF motion element 20″ and the support belt (11) allow motion of the attachment mechanism (44) around the abduction/adduction axis (15) through bending and around the internal/external rotation axis (16) through twisting, while restricting motion in the flexion/extension axis (17).

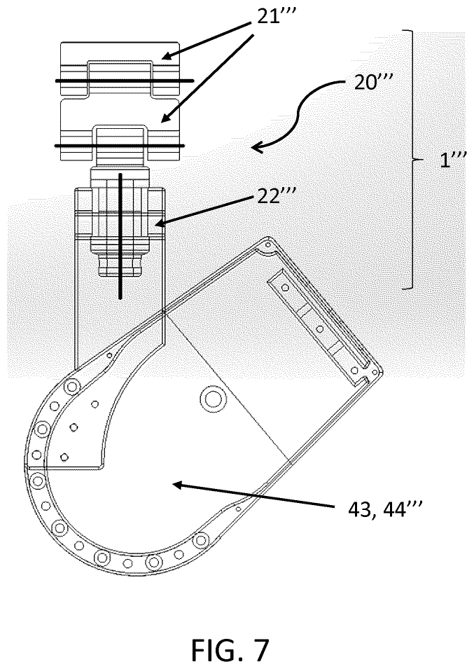

Referring to FIG. 7, there is shown a third alternative embodiment of the full Range of Motion (ROM) and multi-Degree of Freedom (DoF) hip structure (1′″), which includes a frontal adduction (21′″) and an abduction (22′″) DoF mechanisms, and either an interconnection (43) or a hip actuator support (44′″).

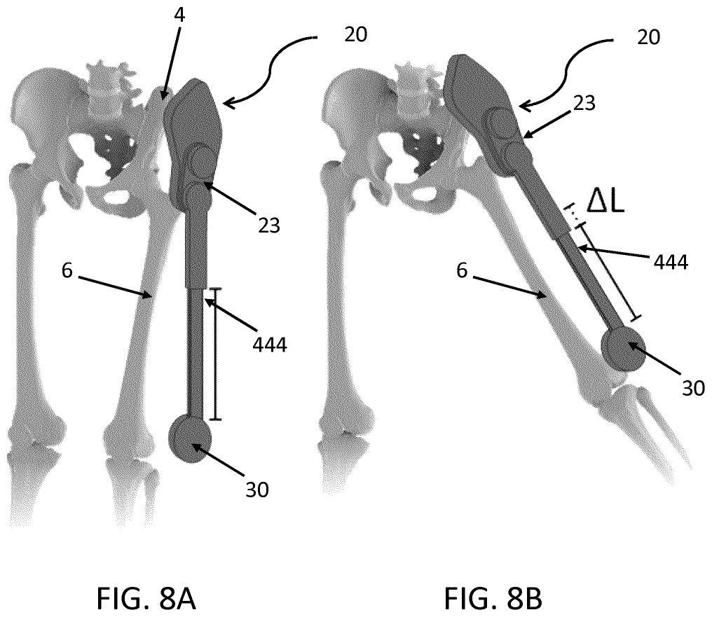

Referring now to FIGS. 8A and 8B, there is shown an alternative embodiment of the attachment mechanism (44) in the form of an off-axis sagittal plane translational DoF mechanism (444), which is a self-adjusting variable length structure, allowing a variation of distance between the horizontal transverse DoF mechanism (23) and the orthotic device (30) so as to prevent misalignment from imposing physical constraints on the movement of the biological joints.

In one illustrative embodiment, the off-axis sagittal plane translational DoF mechanism (444) may take the form of a prismatic joint, for example a dovetail joint and linear bearings. In another illustrative embodiment, the off-axis sagittal plane translational DoF mechanism (444) may take the form of a cylindrical joint, for example an axle on a chassis and a first cylinder sliding into a second cylinder.

Although the present disclosure has been described by way of particular non-limiting illustrative embodiments and examples thereof, it should be noted that it will be apparent to persons skilled in the art that modifications may be applied to the present particular embodiment without departing from the scope of the present disclosure.

1. A compact, multi-Degree of Freedom (DoF) support structure (1) located within a single biomechanical plane, for supporting an orthotic device (30) allowing for a user's full range of motion in all biomechanical plans, the support structure comprising:
a support belt (11) configured to be secured around the user's body, connected to a multi-DoF motion element (20) through a first interconnection (41), the multi-DoF motion element (20) including a first (21), a second (22) and a third (23) rotational DoF mechanisms, the first rotational DoF mechanism (21) being connected to the second rotational DoF mechanism (22) through a second interconnection (42), and the second rotational DoF mechanism (22) being connected to the third rotational DoF mechanism (23) through a third interconnection (43), each of the first (21), second (22) and third (23) rotational DoF mechanisms being uniquely selected from a group consisting of a frontal plane rotational DoF mechanism, a transverse plane rotational DoF mechanism and a sagittal plane rotational DoF mechanism;
the multi-DoF motion element (20) further including an attachment mechanism (44) for connecting thereto an orthotic device (30);
wherein in use the compact, multi-Degree of Freedom (DoF) support structure (1) supports the orthotic device (30) while allowing for the user's full range of motion in all biomechanical plans.

2. The compact, multi-Degree of Freedom (DoF) support structure (1) according to claim 1, wherein the support belt (11) includes a zero-rigidity material.

3. The compact, multi-Degree of Freedom (DoF) support structure (1) according to claim 1, wherein the support belt (11) includes an infinite rigidity material.

4. The compact, multi-Degree of Freedom (DoF) support structure (1) according to claim 1, wherein the support belt (11) includes a material having a rigidity different than zero and infinity.

5. The compact, multi-Degree of Freedom (DoF) support structure (1) according to claim 1, wherein the support belt (11) includes a material selected from the group consisting of fabric, foam, plastic and metal.

6. The compact, multi-Degree of Freedom (DoF) support structure (1) according to claim 1, wherein the frontal rotational DoF mechanism (21), the transverse rotational DoF mechanism (22) and the sagittal rotational DoF mechanism (23) take the form of a mechanism selected from the group consisting of a pivot, a hinge, a passive rotational mechanism and an active rotational mechanism.

7. The compact, multi-Degree of Freedom (DoF) support structure (1) according to claim 1, wherein the frontal rotational DoF mechanism (21), the transverse rotational DoF mechanism (22) and the sagittal rotational DoF mechanism (23) include a zero-rigidity material.

8. The compact, multi-Degree of Freedom (DoF) support structure (1) according to claim 1, wherein the frontal rotational DoF mechanism (21), the transverse rotational DoF mechanism (22) and the sagittal rotational DoF mechanism (23) include an infinite rigidity material.

9. The compact, multi-Degree of Freedom (DoF) support structure (1) according to claim 1, wherein the frontal rotational DoF mechanism (21), the transverse rotational DoF mechanism (22) and the sagittal rotational DoF mechanism (23) include a material having a rigidity different than zero and infinity.

10. The compact, multi-Degree of Freedom (DoF) support structure (1) according to claim 1, wherein the frontal rotational DoF mechanism (21), the transverse rotational DoF mechanism (22) and the sagittal rotational DoF mechanism (23) include a material selected from the group consisting of fabric, foam, plastic and metal.

11. The compact, multi-Degree of Freedom (DoF) support structure (1) according to claim 1, wherein the orthotic device (30) is a passive orthosis.

12. compact, multi-Degree of Freedom (DoF) support structure (1) according to claim 1, wherein the orthotic device (30) is a powered orthosis.

13. The compact, multi-Degree of Freedom (DoF) support structure (1) according to claim 1, wherein the third rotational DoF mechanism (23) is a sagittal plane rotational DoF mechanism, and the attachment mechanism (44) is an off-axis sagittal plane translational DoF mechanism (444) passively linking the third rotational DoF mechanism (23) to the orthotic device (30), wherein the off-axis sagittal plane translational DoF mechanism (444) is a self-adjusting variable length structure allowing a variation of distance between the third rotational DoF mechanism (23) and the orthotic device (30) so as to prevent misalignment from imposing physical constraints on the movement of the biological joints.

14. The compact, multi-Degree of Freedom (DoF) support structure (1) according to claim 13, wherein the off-axis sagittal plane translational DoF mechanism (444) is in the form of a prismatic joint.

15. The compact, multi-Degree of Freedom (DoF) support structure (1) according to claim 13, wherein the off-axis sagittal plane translational DoF mechanism (444) is in the form of a cylindrical joint.

16. The compact, multi-Degree of Freedom (DoF) support structure (1) according to claim 1, wherein the orthotic device (30) includes a joint selected from the group consisting of an ankle joint, a knee joint, a hip joint, a wrist joint, an elbow joint and a shoulder joint, aligned with a corresponding user's joint.

17. The compact, multi-Degree of Freedom (DoF) support structure (1) according to claim 1, wherein the support belt (11) is configured to be secured around the user's torso or waist above the hip joint.

18. The compact, multi-Degree of Freedom (DoF) support structure (1) according to claim 1, wherein the support belt (11) is configured to be secured around the user's shoulders above the shoulder joint.

19. The compact, multi-Degree of Freedom (DoF) support structure (1) according to claim 1, wherein the support belt (11) is configured to be secured around the user's leg above the knee joint.

20. The compact, multi-Degree of Freedom (DoF) support structure (1) according to claim 1, wherein the support belt (11) is configured to be secured around the user's arm above the elbow joint.
